(19) United States
(12) Patent Application Publication
(10) Pub. No.: US 2023/0000503 A1
(43) Pub. Date: Jan. 5, 2023

(54) ORTHOPEDIC BONE CLAMP

(71) Applicant: AlphaMet Ltd, Doar Na Misgav (IL)

(72) Inventor: Rafi HERZOG, Ramat Gan (IL)

(73) Assignee: AlphaMet Ltd, Doar Na Misgav (IL)

(21) Appl. No.: 17/624,395

(22) PCT Filed: Jul. 2, 2020
(86) PCT No.: PCT/IL2020/050743

§ 371 Date: Jan. 3, 2022

Related U.S. Application Data
(60) Provisional application No. 62/870,056, filed on Jul. 3, 2019.

(51) Int. Cl. A61B 17/17; A61B 90/00

(52) U.S. Cl. CPC A61B 17/1775; A61B 90/06; A61B 2090/064; A61B 2017/565

(57) ABSTRACT

A medical device and a method of using same are provided. The medical device includes a first arm and a second arm mounted on a support rail, each of the first arm and the second arm having a bone contacting region. The medical device also includes a mechanism for reducing a distance between the bone contacting regions hereby applying a force to a bone or bones positioned therebetween and a force gauge for indicating a force applied by the bone contacting regions.

### RELATED APPLICATION/S

This application claims the benefit of priority of U.S. Provisional Patent Application No. 62/870,056 filed on Jul. 3, 2019, the contents of which are incorporated herein by reference in their entirety.

### BACKGROUND

The present invention relates to a bone clamp and methods of using same, and more particularly, to a bone clamp which can be used to align a bone or bones and drill one or more holes through a single bone or two adjacent bones. Embodiments of the present invention relate to use of the present bone clamp in treatment of bone deformity disorders such hallux valgus.

Orthopedic procedures used in the repair of broken or deformed bones oftentimes require controlled and accurate realignment of bones and drilling of holes for positioning of fixation devices such as plates, screws, pins and/or wires. Since such implants must be accurately positioned for optimal bone or bone fragment fixation surgeons utilize drill guides to ensure that the drill holes are at a desired position and direction across the bone.

Hallux valgus is a disorder associated with outward deformity (varus) of the 1^{st} metatarsal and inward deformity (valgus) of the hallux with frequent cases of rotation of the 1^{st} metatarsal head. Rotation of the head rotates the sesamoids as well and position them in angle to the transverse plane.

In recent years, a number of minimally invasive approaches have been devised for correcting hallux valgus deformities. These approaches interconnect metatarsal bones under tension in an attempt to restore normal (pre-deformity) bone in the plantar plane and angular position.

In cases of hallux valgus disorders in which the 1^{st} metatarsal is deformed outward (primus varus), button (plates) and suture (wire) type implants are used to correct the deformity without osteotomy. To pass the suture from the medial end of the 1^{st} metatarsal to the lateral end of the 2^{nd} metatarsal (or vs vs) a hole is drilled across the first and second metatarsal bones;

Large diameter holes can cause bone stress fracture especially in the 2^{nd} metatarsal bone which is of smaller diameter (typically 7-9 mm). For example, a fixation device that uses 2.7 mm diameter hole increases the probability of stress fracture on 2^{nd} metatarsal (30-40% are reported). As a result, it is desired to drill the 2^{nd} metatarsal with a smaller diameter holes in the range of 1.1-1.8 mm.

Due the distance and outward deformity, when drilling small diameter holes across the 1^{st} and 2^{nd} metatarsals the drill bit can bend/deflect such that the resulting hole in the second bone can be misaligned with the hole in the first bone thereby leading to misaligned anchoring.

Thus it would be highly advantageous to have a medical device that can approximate and align two adjacent bones for drilling such that subsequent fixation of the bones is aligned along a desired and predetermined plane/path.

### SUMMARY

According to one aspect of the present invention there is provided a medical device comprising a first arm and a second arm mounted on a support rail, each of the first arm and the second arm having a bone contacting region; a mechanism for reducing a distance between the bone contacting regions of the first arm and the second arm thereby applying a clamping force to a bone or bones, positioned between the bone contacting regions; and a force gauge for indicating the force applied by the bone contacting regions to the bone or bones. Removable drill leads attached to the arms at the contacting regions guide the drill bits at the desired trajectory to create a single line or single plane drill path.

According to another aspect of the present invention there is provided a method of correcting hallux valgus in a subject comprising positioning a stiff small diameter wire (such as K-wire) through a first metatarsal and a second metatarsal; positioning the device over the wire; clamping the first metatarsal and the second metatarsal between the bone contacting regions of the device; reducing a distance between the bone contacting regions of the first arm and the second arm until a predetermined force is achieved as indicated by the force gauge and/or a desired angle is achieved between the bones; drilling holes in the first metatarsal and the second metatarsal using pre-attached drill leads (each bone is drilled separately from different direction forming one line), passing through the wires of the fixation device, removing the drill leads (only) to free the “work” area’, positioning the fixation device into the bone and plates over the bone surface and then locking/securing the wires of the fixation device. After the fixation device is secured the clamping device is removed.

According to another aspect of the present invention there is provided a bone fixation device comprising two anchors each having an anchor body positionable within a bone and a flange having an opening and a mechanism for locking a wire/suture/thread therein.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE SEVERAL VIEWS OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1A-B illustrate one embodiment of the bone clamp of the present invention.

FIG. 2 illustrates a disassembled state of the bone clamp of FIGS. 1A-B.

FIGS. 3A-C illustrate two adjacent bones clamped by the bone contacting regions of the bone clamp of FIGS. 1A-B and the K-wires and drill bits.

FIG. 4 illustrates the device with the drill leads removed.

FIG. 5 illustrates the offset angle of the first and second arms with respect to the bone contacting regions and the angle of drilling.

FIGS. 6A-B illustrate one embodiment of a bone fixation device utilizable with the present invention.

FIG. 7 illustrates the fixation device with a pass through insertion element.

FIG. 8 illustrate the bone fixation device positioned across the first and second metatarsals.

### DETAILED DESCRIPTION

The present invention is of a bone clamp which can be used to align adjacent bones and drill these bones to form a common drill plane or common drill line.

The principles and operation of the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

In Hallux Valgus correction surgery, the 1^{st} metatarsal (which is deformed outward) is pushed back towards the 2^{nd} metatarsal in order to reduce the angle between the first and second metatarsals [known as Intermetatarsal angle (IMA)] to an acceptable normal value (medically accepted values are less than 9°—Campbell's Operative Orthopedics). In those surgical methods that avoid any bone osteotomy, the bone is pushed inward by applying force on the 1^{st} metatarsal bone and a button and suture fixation device holds the 1^{st} bone at the desired position (IMA smaller than 9°, typically 4-7°). The force required for this realignment is about 25-40 N. This procedure necessitates holding the bones during surgery in an anatomically correct position and simultaneously drilling a correct path through the bones for the implant. As such, surgeons often rely upon assistants to hold the bones. Since bone holding and drilling are performed manually the results can be inconsistent and lead to poor fixation.

The present inventor devised a medical device that can be used to controllably clamp adjacent bones while compensating for bone relocation and rotation during deformity correction. The present device can also be used to guide drilling of holes through two adjacent bones at different trajectories and different directions such that the holes are aligned through a common plane/line and an implant (fixation device) secured therethrough fixates the bones at a desired IMA.

Thus, according to one aspect of the present invention there is provided a medical device that can be used to clamp and optionally drill holes in a bone or bones. The medical device of the present invention can be used to clamp any bone in repair of bone trauma (e.g., fracture) or disorder (e.g., bone deformity such as Hallux valgus). The medical (orthopedic) device (also referred to herein as “bone clamp”) can also be used to guide one or more drill bits in drilling through one or more bones. The device of the present invention can be positioned externally (outside the skin) or internally within the body after exposing the bone and removing soft tissues.

The medical device includes a first arm and a second arm mounted on a support rail (approximately midway along the length of the arms), each arm includes a bone contacting region at a bottom end of the arm and an alignment hole for mounting over a stiff wire (e.g., K-wire). The support rail can be attached to one arm with the other arm movable along its length. The medical device further includes a mechanism for reducing a distance between the bone contacting regions of the arms thereby clamping the bone or bones positioned between the bone contacting regions. The arm movable along the support rail (e.g., second arm) can engage the rail via a ratchet mechanism. Such a mechanism allows stepwise movement of the arm along the rail and can include a release button for releasing the arm (sliding it off the support rail). The ratchet mechanism can include teeth on the support rail and a spring loaded tab for engaging the space between teeth in the second arm. The release button can move the tab out of engagement with the teeth. The ratchet mechanism can be used to first place the device over a bone or bones manually setting a distance and perform initial clamping action. A second mechanism for further reducing the distance between the bone contacting regions of the arms can then be used to apply an inward clamping force (squeezing) on the bone or bones (approximating bone contacting regions). A force gauge mounted on the device can indicate the force applied to the bones by the bone contacting regions of the arms.

The medical device can also include a second (guide) rail for stabilizing the arms (when applying a force to the bones) to prevent torsion or buckling of the arms. Such a guide rail can be fixed to the first arm and slidably engage the second arm through an opening at the top of the arm.

The mechanism for reducing a distance between the bone contacting regions of the arms can include a turn knob mounted over a threaded bar or bars. Turning the knob moves a lower portion of the second arm towards the first arm.

The bone contacting regions of the arm can be configured for attachment of drill guide adaptors (also referred to herein as “drill leads”). Such drill leads can include one or more (e.g., 2, 3, 4) drill holes/guides for facilitating drilling of holes in the bones clamped by the present device. The drill leads are attachable and fixed to the arms. Each drill lead can be independently angled (factory set or following attachment by surgeon to enable drilling at different angles with respect to the bone or bones.

The bone contacting region has a horizontal bar attached to it. The bar provides reference to the angular position of the device (“Leveling bar”). The leveling ensures that when more than one hole is drilled in each bone, the holes will be at a desired axis. When the present device is clamped and leveled by the leveling bar along the bone dorsal surface the arms and rails are angled (about 30°) in a way that the device does not obscure the surgeon's view of the bones and enables X-ray imaging when required.

The present invention is particularly suitable for correction of bone deformities such as 1^{st} metatarsal primus varus by providing a bone clamp that can compensate for bone relocation and rotation during deformity correction using a fixation device such as button and suture device.

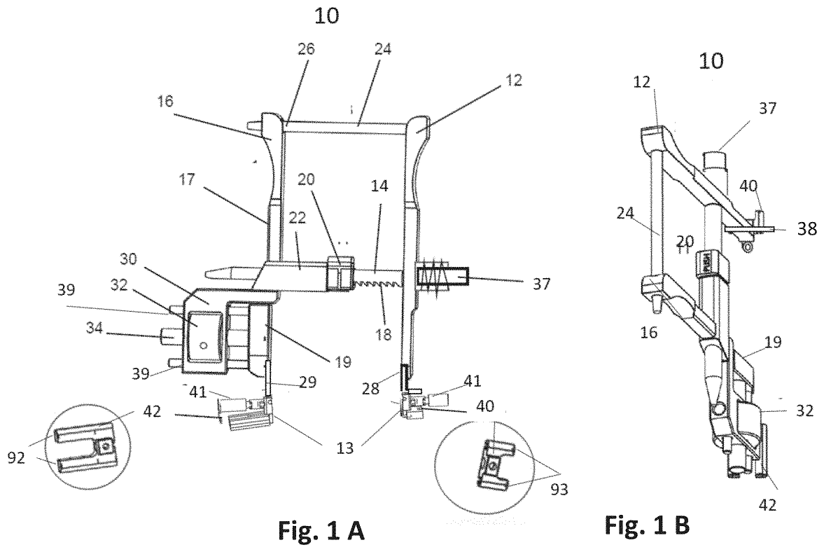

Referring now to the drawings, FIGS. 1A-8 illustrate an embodiment of the present bone clamp (referred to hereinunder as device 10) which is suitable for use in Hallux valgus repair. FIGS. 1A-B illustrate front (FIG. 1A) and isometric (FIG. 1B) views of device 10.

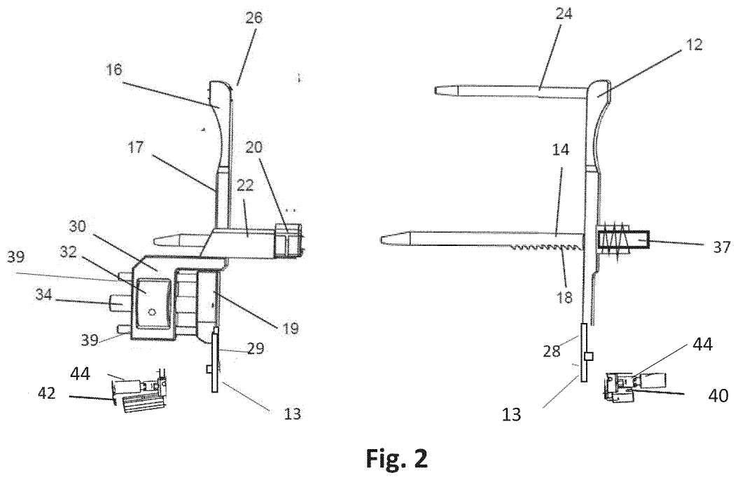

FIG. 2 illustrates device 10 in a disassembled state in which arms 12 and 16 and drill leads 40 and 42 are detached. Device 10 includes a first arm 12 attached to support rail 14 and a second arm 16 that is movable along support rail 14. Support rail 14 includes teeth 18 that engage a tab (not shown) in second arm 16. This engagement forms a ratchet mechanism that is releasable via push button 20. Second arm 16 can include a bracket 22 (extending in the direction of first arm 12) for stabilizing support rail 14 therewithin. This ensures that forces applied by the tensioning mechanism on the bone or bones do not buckle or deform device 10.

A second rail 24 (guide rail) further supports device 10. Rail 24 is attached to first arm 12 and is slidably engaged with second arm 16 through opening 26.

Arms 12 and 16 each include a bone contacting region 28 and 29 (respectively) that are configured for contacting a bone. Bone contacting regions 28 and 29 are angled (about 30°) with respect to arms 12 and 16. The bone contacting surface of regions 28 and 29 can be roughened or can include teeth or serrations for facilitating engagement. Arms 12 and 16 can each include holes 13 for accepting a K-wire drilled as a reference guide into the bone or bones.

The dimensions of device 10 are as follows, arm 12, 80-120 mm in length and 5-10 mm in width, arm 16 80-120 mm in length and 5-10 mm in width. Bone contacting regions 28 are 10-20 mm in length. Support rail 24 can be 40-70 mm in length and 3-8 mm in width. Device 10 can be fabricated from a medical grade metal, alloy or polymer.

Device 10 further includes a mechanism 30 for tensioning (moving) a lower portion 19 of arm 16 towards arm 12. Arm 16 is split into two portions, an upper portion 17 and lower portions 19 and 30. Upper portion 17 and lower portions 19 and 30 move along support rail 14 and rail 24. Lower portion 19 moves independently of upper portion 17 via mechanism 32 and 34. Small diameter support rails 29 hold arm 19 in vertical position along its movement.

Mechanism 30 includes a turn knob 32 and a threaded bar 34. Turning of knob 32 in a clockwise direction can move bar 34 along knob 32 and thus move lower portion 19 of arm 16 inward (towards arm 12).

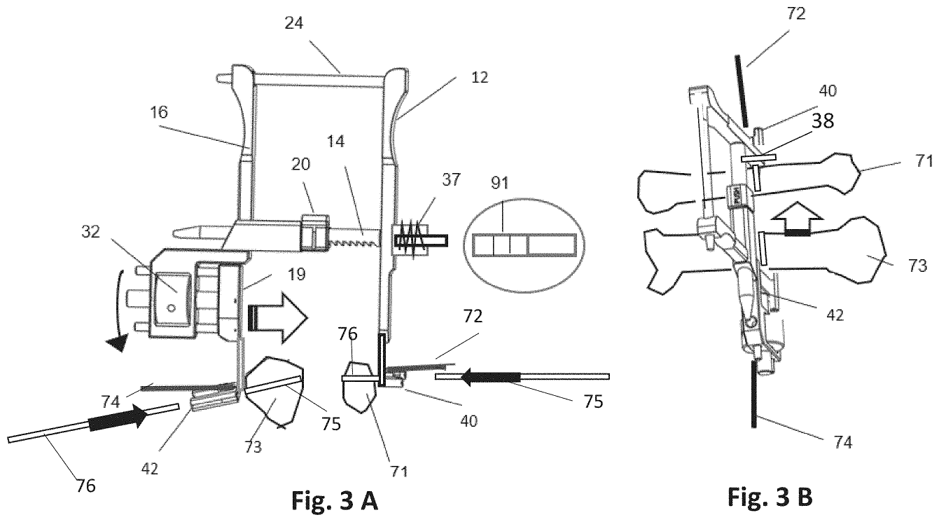

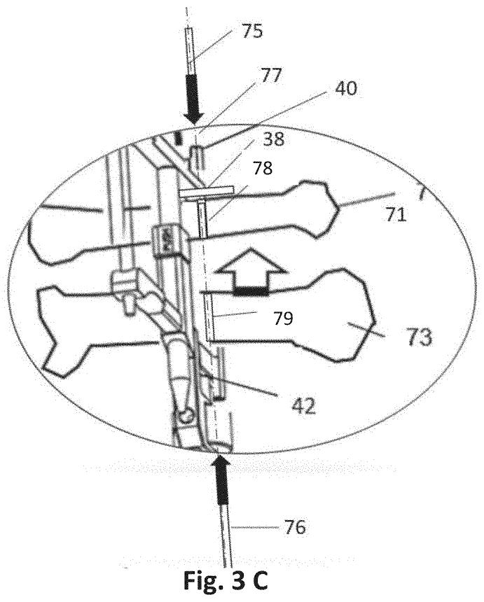

Device 10 further includes a force gauge 37 (load cell, spring, mechanical or electrical) for determining a force applied to bones clamped between bone contacting regions 28 (FIGS. 3A-C). The present inventor has determined that in Hallux valgus repair bone alignment requires a force of 25-35 Newtons (N). As such, the force gauge includes a display 91 for displaying such a range of forces (e.g., 0-40 N). The force gage may be preloaded to certain loads for example to 25 N and as such will only move in response to forces above 25 N.

FIG. 3A-B illustrates side view of the device 10 clamping the bones as well as K-wires 72 and 74 for positioning and stabilizing the device on the bones. Drill bits 75 and 78 are shown aimed in the direction of drill leads 40 and 42.

FIG. 3C illustrates a top view of device 10 positioned over the bones, drill bit direction and the drilled holes in the bones. Hole 78 in 2^{nd} bone 71 is drilled by drill bit 75 using drill lead 40. Hole 79 in 1^{st} bone 73 is drilled by drill bit 76 using drill lead 42. Holes 78 and 79 are aligned along line 77 on the plantar plane. Holes 78 and 79 are drilled independently and can have different diameters. For example, 2^{nd} bone hole 78 can be 1.1-1.8 mm in diameter and 1^{st} bone hole 79 can be 2-3 mm in diameter.

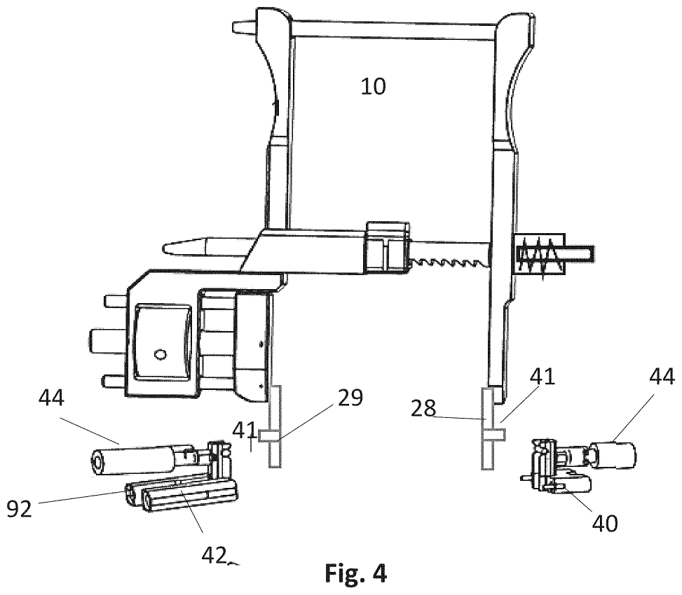

FIG. 4 illustrates device 10 and drill leads 40 and 42 each attachable via thread mechanism 44 to bone contacting regions 28 and 29 at a guiding screw 41. Drill lead 40 has a male-female connector to ensure attachment to the correct arm. Drill lead 40 can be attached and fixed to bone contacting region 28 only and drill lead 42 can be similarly attached and fixed to bone contacting region 29 only. Drill lead 40 includes guide holes that are spaced 8-12 mm apart with each having a bore 93 1.5-3 mm in diameter. Drill lead 42 includes two guide holes that are spaced apart 8-12 mm with each having a bore 92 1.5-3 mm in diameter (both guide holes at drill leads 40 and 42 can be equally spaced apart). Drill lead 40 has a length of 6-15 mm and drill lead 42 has a length of 6-15 mm. The length of each drill lead is determined by anatomical limitations and required field of view.

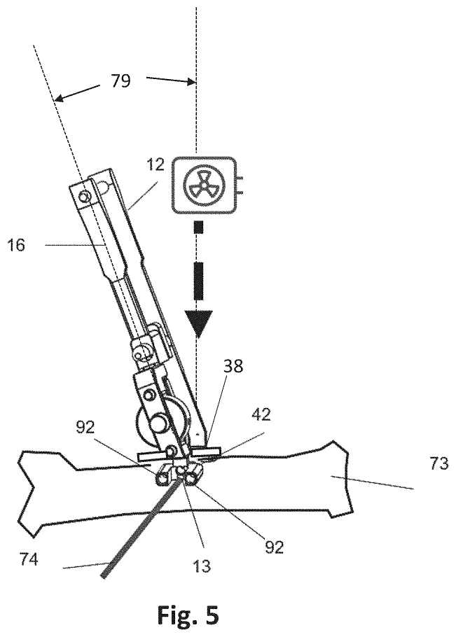

FIG. 5 shows device 10 leveled by the leveling bar 38. Since device 10 is used in a procedure that requires also periodic imaging, it is configured such that when clamped over a bone or bones and leveled, the bulk of device 10 is angled away 79 from the plane of imaging.

As is mentioned herein above, device 10 is used to clamp adjacent bones in a desired position for drilling in order to position a bone fixation device through the bone and perform the required drilling bores.

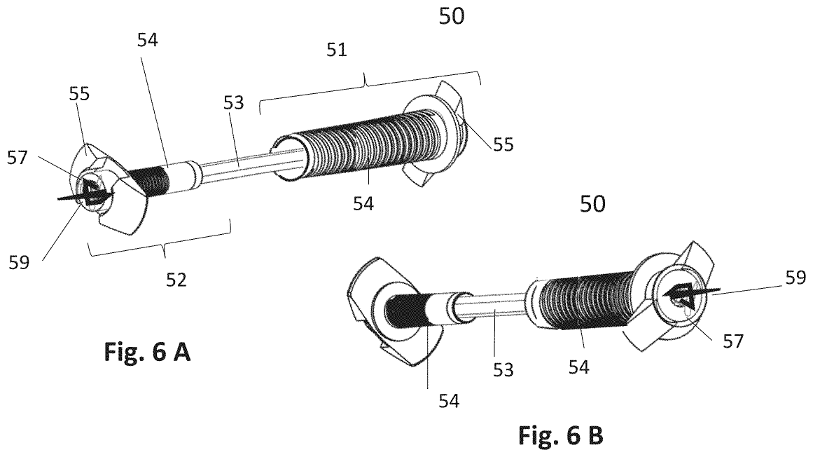

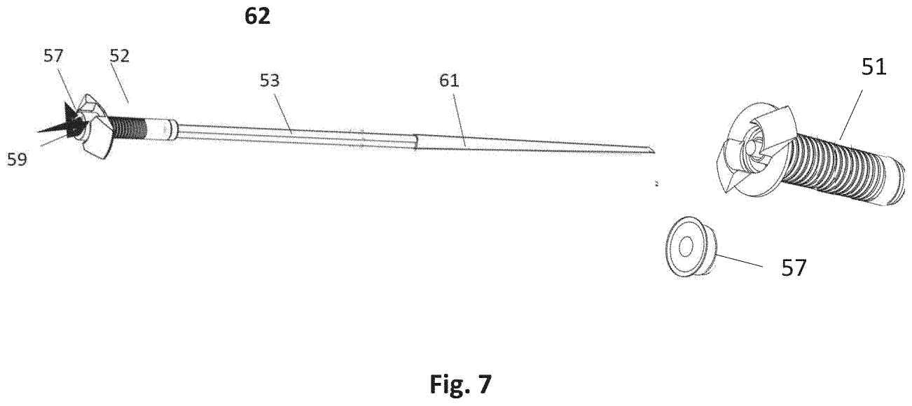

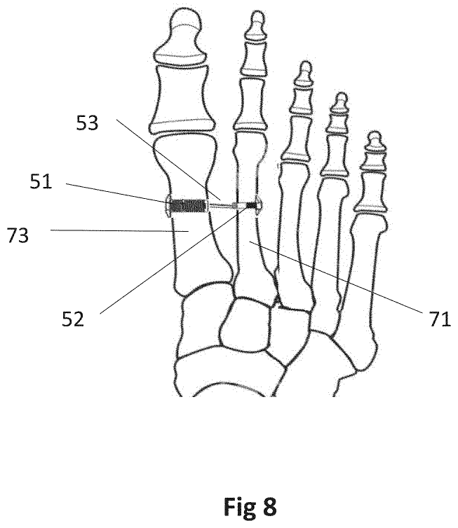

FIGS. 6A-B illustrate one embodiment of a bone fixation device which is referred to herein as fixation device 50. FIG. 7 illustrates fixation device 50 ready for implantation. FIG. 8 illustrates a fully implanted fixation device 50 post-surgery.

Fixation device 50 includes two bone anchors 51 and 52 that can have different diameters to fit bone drill hole diameter. First metatarsal anchor 51 can have a diameter of 2-3 mm. Second metatarsal, which is smaller in diameter has anchor 52 with diameter of 1.5-2 mm. Anchors 51 and 52 are connectable via a connecting element 53 that can be a wire, suture, thread braid and the like (“wire” is used here as a general term) (FIGS. 6A-B).

Each of anchors 51 and 52 includes an anchor body 54 with threaded/partially threaded or smooth outers surface and a flange 55 that abuts the bone surface when body 54 is positioned within the bone drill hole. Connecting element 53 is threaded through body 54 and is secured against flange 55 using a looped wire or wire with knot (on one size) and a knot (on the opposing side).

FIG. 7 illustrates the fixation device as provided to surgeons. Device 50 has three components, a pre-assembled component 62 that includes anchor 52, small diameter ring-button 57 and wire 53 secured by a knot. Wire 53 is collated into a wire tube (“wire lead”) with a small diameter or pointed end. Component two includes anchor 51 and component three is ring-button 57.

Positioning of fixation device 50 across adjacent metatarsals (M1 and M2) is shown in FIG. 8.

Device 10 of the present invention can be used for Hallux valgus correction as follows:

The surgeon determines the location of the implant in the 1^{st} metatarsal (1^{st} bone) and 2^{nd} metatarsal (2^{nd} bone) bones—generally at midshaft of these bones. The surgeon then performs incisions at implantation area, a medial incision in the first metatarsal and a lateral incision at the second metatarsal thereby exposing the bone area. K-wires 73, 74 are then drilled into in each bone, generally at first metatarsal below bone center line and at the second metatarsal about 1 mm above the center line. The K-wires serve as a positioning lock for the present medical device 10.

Prior to positioning arms 12 and 16 over the K-wire, drill leads 40 and 42 are attached to the arms and locked firm thereto.

Arms 12, 16 are then positioned over the K-wires using holes 13. While sliding the arms over the K-wire the two parts are attached together (rail 14 into bracket 22, rail 24 into hole 26) and pushed towards each other and clamped manually (ratchet 18 locks arms 12 and 16 in position).

Knob 32 is then rotated clockwise to approximate the bones. The force applied on the bones is indicated on the force gage. The distance is reduced until a satisfactory inter metatarsal angle (IMA) between the bones is achieved (typically 5-7°), or a maximal allowed force (45 N) is indicated on force gauge 39.

The bones are then drilled by drills 75, 76 through drill leads 40 and 42, each bone is drilled separately, and each bone can be drilled via a different diameter drill bit; in the 1st metatarsal—medial to lateral and in the 2^{nd} metatarsal—lateral to medial. The implant wires are then passed through the holes from the 2^{nd} metatarsal to the 1^{st} metatarsal using a wire lead 61 which is attached to the wires (FIG. 7). Drill leads 40 and 42 are then removed from arms 28 and 29 and the wires are then completely passed through the bones. The wire lead is pulled out and detached from the wires. Anchor 52 is than pushed into the bone and screwed therein to a position where flange 55 abuts the bone. The wires are then threaded through the second anchor 51 (which can have a larger opening through body 54 in order to facilitate smooth and easy wire threading) and then pushed partially into the bone holes and screwed therein. A ring 57 is than positioned over the wires and pulled to the anchor surface. Button 57 is then crimped to reduce its diameter and the wires are secured via multiple knots.

As used herein the term “about” refers to ±10%.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

In addition, any priority document(s) of this application is/are hereby incorporated herein by reference in its/their entirety.

What is claimed is:

1. A medical device comprising:
(a) a first arm and a second arm mounted on a support rail, each of said first arm and said second arm having a bone contacting region;
(b) a mechanism for reducing a distance between said bone contacting regions of said first arm and said second arm thereby applying a force to a bone or bones positioned between said bone contacting regions; and
(c) a force gauge for indicating a force applied by said bone contacting regions to said bone or bones.

2. The medical device of claim 1, wherein said second arm interfaces with said support rail through a ratchet mechanism.

3. The medical device of claim 2, wherein said mechanism moves a portion of said second arm towards said first arm along said support rail.

4. The medical device of claim 2, wherein said mechanism is attached to said second arm and includes a rotatable knob for moving said second arm towards said first arm.

5. The medical device of claim 3, wherein said ratchet mechanism includes a release button for enabling free sliding of said second arm on said support rail.

6. The medical device of claim 1, further comprising drill leads attachable to said first arm and said second arm.

7. The medical device of claim 6, wherein each of said drill leads includes one or more spaced apart drill hole guides.

8. The medical device of claim 6, wherein said drill leads can be independently angled to enable drilling at different angles with respect to said bone or bones.

9. The medical device of claim 6, wherein said bone contacting regions are angled with respect to said first and said second arms.

10. The medical device of claim 1, wherein said support rail forms a part of said first arm and said second arm is movable along said support rail.

11. The medical device of claim 1, further comprising a guide rail interconnecting said first arm and said second arm.

12. The medical device of claim 11, wherein said guide rail forms a part of said first arm and said second arm is movable along said guide rail.

13. The medical device of claim 4, wherein said force gauge is mounted on said first arm.

14. The medical device of claim 6, wherein each of said first and said second arms includes an alignment hole.

15. The medical device of claim 7, wherein each of said drill leads includes guide holes of different diameter.

16. A method of correcting hallux valgus in a subject comprising:
(a) positioning wires in a first metatarsal and a second metatarsal;
(b) positioning the device of claim 1 over said wires;
(c) clamping said first metatarsal and said second metatarsal between said bone contacting regions of the device;
(d) reducing a distance between said bone contacting regions of said first arm and said second arm until a predetermined force is achieved as indicated by said force gauge and/or a desired angle is achieved between the bones;
(e) drilling holes in the said first metatarsal and said second metatarsal using drill leads attached to said device;
(f) inserting a first bone anchor through a drill hole in said first metatarsal;
(g) removing said drill leads;
(h) inserting a second bone anchor through a drill hole in said second metatarsal;
(i) interconnecting said first and said second anchors via a wire; and
(j) removing the clamping device.
